(19) United States
(12) Patent Application Publication
(10) Pub. No.: US 2007/0179137 A1
(43) Pub. Date: Aug. 2, 2007

(54) Screening of anti-viral drugs and pharmaceuticals composition containing thiazolidinone derivatives

(75) Inventors: Paul Gregor, Rehovot (IL); Nicholas Harris, Rehovot (IL); Regina Zhuk, Ness Ziona (IL)

Correspondence Address: BROWDY AND NEIMARK, P.L.L.C.;624 NINTH STREET, NW, SUITE 300, WASHINGTON, DC, 20001-5303, US

(73) Assignee: RIMONYX PHARMACEUTICALS LTD., NESS-ZIONA (IL)

(21) Appl. No.: 10/594,080

(22) PCT Filed: Mar. 24, 2005
(86) PCT No.: PCT/IL05/00335

§ 371 Date: Sep. 25, 2006

Related U.S. Application Data
(60) Provisional application No. 60/555,667, filed on Mar. 24, 2004.

(51) Int. Cl. A61K 31/5377; A61K 31/519; A61K 31/427; C07D 487/04; C07D 417/02; C12Q 1/70

(52) U.S. Cl. 514/234.2; 514/259.41; 544/279; 514/252.16; 544/114; 435/5; 548/181; 514/369

(57) ABSTRACT

The present invention relates to a method of screening for small organic molecules that directly inhibit the interaction of glycosaminoglycans (GAGs) with GAG-binding viral proteins (GBVPs), which comprises contacting a GAG with an GBVP in the presence of at least one candidate compound; and measuring the amount of the GAG bound to the GBVP or the amount of the GBVP bound to the GAG, wherein a significant decrease in GAG-GBVP binding as compared to GAG-GBVP binding in the absence of the candidate compound, identifies said compound as inhibitor of the GAG-GBVP interaction. The invention further provides pharmaceutical compositions comprising certain 2-thioxo-thiazolidinone derivatives, particularly useful against virus infections.

### FIELD OF THE INVENTION

The present invention relates in general to the field of drug screening, more particularly to screening of drugs for the treatment of diseases that involve deleterious virus-cell adhesion, virus attachment, virus entry and virus infection. Specifically, the present invention relates to methods for screening, identification and optimization of small organic molecules that inhibit virus attachment to cells mediated by glycosaminoglycans (GAGs), and use thereof for the treatment of viral diseases. The present invention relates also to pharmaceutical composition comprising compounds capable of inhibiting the interactions between GAGs, particularly heparan sulfate glycosaminoglycans (HS-GAGs), and GAG-binding viral proteins (GBVPs).

Abbreviations: ACV: acyclovir; BSA: bovine serum albumin; CMV: human cytomegalovirus; CPE: cytopathic effect; ECM: extracellular matrix; FMDV: foot-and-mouth disease virus; GAGs: glycosaminoglycans; GBVPs: GAG-binding viral proteins; gC-2: glycoprotein C of HSV-2; HCV: hepatitis C virus; HFF: human foreskin fibroblast; HHV-7: human herpes virus 7; HIV-1: human immunodeficiency virus type 1; HS: heparan sulfate; HS-PGs/HSPGs: heparan sulfate proteoglycans; HSV-1: herpes simplex virus type 1; RSV: respiratory syncytial virus; VZV: varicella-zoster virus.

### BACKGROUND OF THE INVENTION

The extracellular matrix (ECM) has an important function in providing structural integrity to tissues and in presenting appropriate environmental cues for cell adhesion, migration, growth, and differentiation. Major constituents of ECM include glycosaminoglycans (GAGs), fibronectin, laminin, collagen and proteoglycans, which mediate and drive specific cell surface receptor-ligand interactions. Many protozoa, bacteria and viruses have been shown to bind cell surface GAGs. Viruses have evolved to exploit the cell-surface GAGs, particularly heparan sulfate (HS)-GAGs, to facilitate their attachment and infection of host cells. A growing body of evidence points to the role of cell surface HS-GAGs as the initial receptor in viral infection.

Glycosaminoglycans Glycosaminoglycans (also referred to herein and in the art as “GAG” or “GAGs”) are naturally-occurring carbohydrate-based molecules implicated in the regulation of a number of cellular processes, including blood coagulation, angiogenesis, tumor growth and smooth muscle cell proliferation, most likely by interaction with effector molecules. GAGs are linear, non-branched chains of repeating two-sugar (disaccharide) units, which may be up to 150 units in length.

Glycosaminoglycans can be divided into four main classes on the basis of a repeating disaccharide unit in the backbone. Typically, one sugar is an uronic acid, and the other is either an N-acetylglucosamine or an N-acetylgalactosamine. The classes are exemplified by the following GAGs: (1) heparan sulfate (D-glucuronic acid/N-acetyl- or N-sulfo-D-glucosamine); (2) chondroitin/dermatan sulfate (D-glucuronic acid or L-iduronic acid/N-acetyl-D-galactosamine); (3) keratan sulfate (D-galactose/N-acetyl-D-glucosamine); and (4) hyaluronic acid (glucuronic acid/N-acetyl-D-glucosamine). All GAGs (with the exception of hyaluronic acid) contain sulfate groups variously esterified to the ring hydroxyl groups of the sugars. These negatively charged groups are believed to figure prominently in the biological properties attributed to glycosaminoglycans. The naturally occurring forms of GAGs, particularly heparin, heparan sulfate, chondroitin sulfate and dermatan sulfate are, in fact, complex hetero-oligosaccharides composed of mixtures of differentially sulfated sugar residues.

One of the most thoroughly studied glycosaminoglycans is the widely used anticoagulant heparin. Heparin, a highly sulfated form of heparan sulfate, is found in mast cells. Overall, heparin is less abundant than related sulfated polysaccharides, such as heparan sulfate, dermatan sulfate, and chondroitin sulfate, which are synthesized in nearly all tissues of vertebrates. As a commercial product, heparin is a hetero-oligodisaccharide composition of about 20-60 monomeric units.

Heparan sulfate glycosaminoglycans (also referred to herein and in the art as “HS-GAGs”) consist of repeating disaccharide units. Relatively small segments of HS-GAGs contain disaccharide units that are the actual binding sites for ligands (usually 3-10 disaccharides out of 40-160 disaccharides). The specificity of the GAG biosynthetic enzymes imposes restrictions on the disaccharide GAG sequence. HS-GAG chains typically contain regions rich in GlcA and GlcNAc (N-acetylated domains), contiguous variable length sequences containing GlcNS derivatives (N-sulfated domains), and some sections that contain alternating N-acetylated and N-sulfated units of glucosamine. Typical HS-GAG chains contain relatively short segments of modified sequences interspersed among large sections of unmodified units. Interestingly, the relative content of N-acetylated, N-sulfated, and N-acetylated/N-sulfated domains as well as other properties of the chains appears to be a stable characteristic of the cells from which the HS-GAG was obtained (Esko J D and Selleck S B 2002 Annu. Rev. Biochem. 71, 435-71).

HS-GAG chains are assembled while they are attached to a proteoglycan core protein. Heparan sulfate proteoglycans (HS-PGs) are ubiquitous macromolecules associated with the cell surface and the ECM of a wide range of cells of vertebrate and invertebrate tissues. The basic HS-PG structure consists of a protein core to which several linear heparan sulfate chains are covalently attached. Three major families of proteoglycan core proteins have been characterized: the membrane-spanning syndecans (four members), the glycosylphosphatidylinositol-linked glypicans (six members), and the basement membrane PGs perlecan and aggrin. Several other HS-GAG-bearing proteoglycans are known as well (e.g., betaglycan and a CD44 splice variant). The syndecans can contain up to five GAG chains whereas glypicans typically contain one to three HS chains. The different core proteins are expressed in a cell-type-specific manner. Studies on the involvement of ECM molecules in cell attachment, growth and differentiation revealed a central role of HS-PGs in embryonic morphogenesis, angiogenesis, metastasis, neurite outgrowth and tissue repair.

Infectious Viruses Sttach to and Enter Cells Via Binding to GAGs

A growing body of evidence points to the role of cell surface GAGs as the initial receptor in viral infection. Specifically, viruses such as herpes simplex virus (HSV), dengue virus, respiratory syncytial virus, varicella-zoster virus (VZV), cytomegalovirus (CMV), cindbis virus, adeno-associated virus, vaccinia virus, foot-and-mouth disease virus (FMDV) and human immunodeficiency virus type 1 (HIV-1) (Chen, Y. et al., 1997, Nature Med. 3, 866-871) all employ HS-GAGs for their initial step of infection. The infectivity of these viruses may be inhibited in vitro by pretreatment of cultured cells with heparitinase prior to the infection, or by addition of exogenous GAGs such as heparin or HS-GAG, or by addition of polysulfated compounds such as suramin (Chen et al., 1997, ibid). The crystal structure of the complex between foot-and-mouth disease virus and HS has been elucidated. Although the mechanism of attachment has been elucidated in only a few cases, it is probably mediated by electrostatic interactions between basic protein domains (e.g., heparin-binding domains) on the surface of viruses and the sulfated HSPG side chains. In the case of HSV-1 entry, an essential role for 3-O-sulfated glucosamine residues on HS has been demonstrated (Shukla D. et al., 1999, Cell 99, 13-22). This indicates that the interactions between viral proteins and HS are very specific.

Specific negatively charged regions of the HS-GAGs, including N-sulfations and the carboxyl groups, are key structural features for interactions of both HSV-1 and HSV-2 with cell surfaces since N-desulfation or carboxyl reduction of heparin abolished its antiviral activity. In contrast, 6-O sulfations and 2-3-O sulfations are important determinants primarily for HSV-1 infection. The O-desulfated heparins had little or no inhibitory effect on HSV-1 infection but inhibited HSV-2 infection. It was found that susceptibility to O-desulfated heparins can be transferred to HSV-1 by the gene for glycoprotein C of HSV-2 (gC-2). This supports the notion that the envelope glycoproteins of HSV-1 and HSV-2 interact with different affinities for different structural features of heparin. To determine if the modified heparin compounds inhibited plaque formation by competing with cell surface HS-GAG for viral attachment, binding studies were also performed. As anticipated, most compounds inhibited binding and plaque formation in parallel, but subtle differences were determined. These results suggest differences in the interactions of HSV-1 and HSV-2 with cell surface HS-GAG that may influence cell tropism. It was confirmed recently that wild-type strains of HSV-1 bind to GAGs. Viruses derived from clinical specimens were, similar to their cell culture propagated progeny viruses, sensitive to heparin. In addition, wild-type HSV-1 infection of HS deficient cells was also impaired (Trybala E et al, 2002, Virology 302:413-9).

After causing childhood chickenpox, VZV remains latent in nerve tissues, held in check by the immune system. It remains there until the immune system is compromised, perhaps as a consequence of stress, illness or just old age. At such times, the virus may reactivate causing shingles particularly in the elderly and presenting as a painful strip of vesicles, usually around the trunk, with the risk of severe, chronic pain and persistent after-effects. VZV causes shingles outbreaks on 3.5 million sufferers in the West each year, with as many as 95% of the world's adult population carrying VZV. The risk of contracting shingles increases with age. There is a shortage of efficient drugs for VZV infections. Current antiviral pharmaceuticals include acyclovir and valaciclovir, which exert an effect on the vesicles that erupt, but only a marginal effect on chronic pain. A broad selection of analgesic drugs and pain killers are used against the chronic pain, with varying results.

GAG-binding Viral Proteins (GBVPs)

Recent data have identified virus-specific proteins which interact with host HS-GAGs. The first example, herpes simplex virus type 1 (HSV-1), binds to cells through interactions of viral glycoproteins gB and gC with heparan sulfate chains of cell surface proteoglycans (Laquerre et al, 1998 J Virol. 72:6119-30). This binding is necessary, but not sufficient for viral entry, which requires fusion between the viral envelope and cell membrane. It was shown that HS-GAG, modified by a subset of the multiple D-glucosaminyl 3-O-sulfotransferase isoforms, provides sites for the binding of a third viral glycoprotein, gD. The interaction between gD and its receptor may stabilize the virus-cell complex prior to membrane fusion which is mediated by other essential glycoproteins such as gB, gH, and gL (Tal-Singer et al 1995, J Virol. 69:4471-4483) and for initiation of HSV-1 entry. It was concluded that the susceptibility of cells to HSV-1 entry depends on (1) the presence of heparan sulfate chains to which virus can bind and (2) 3-O-sulfation of specific glucosamine residues in heparan sulfate to generate gD-binding sites or the expression of other previously identified gD-binding receptors.

HS-GAG has an important role in cell entry by foot-and-mouth disease virus (FMDV). Subtype O1 FMDV binds this GAG with high affinity by immobilizing a specific highly abundant motif of sulfated sugars. The binding site is a shallow depression on the virion surface, located at the junction of the three major capsid proteins, VP1, VP2 and VP3. Two pre-formed sulfate-binding sites control receptor specificity. Residue 56 of VP3, an arginine in this virus, is critical to this recognition, forming a key component of both sites. This residue is a histidine in field isolates of the virus, switching to an arginine in adaptation to tissue culture, forming the high affinity heparan sulfate-binding site. It is postulated that this site is a conserved feature of FMDVs, such that in the infected animal there is a biological advantage to low affinity, more selective, interactions with GAG receptors.

In an attempt to identify the human herpes virus 7 (HHV-7) envelope protein(s) involved in cell surface binding, the extracellular domain of the HHV-7 glycoprotein B (gB) homolog protein was cloned and expressed as a fusion product with the Fc domain of human immunoglobulin G heavy chain gammal (gB-Fc) in a eukaryotic cell system (Secchiero P et al 1997, J Virol 71:4571-80). Indirect immunofluorescence followed by flow cytometric analysis revealed specific binding of gB-Fc to the membrane of SupTi cells but not to other CD4+ T-lymphoblastoid cell lines, such as Jurkat or PM1, clearly indicating that gB-Fc did not bind to the CD4 molecule. This was also suggested by the ability of gB-Fc to bind to CD4-negative fibroblastoid Chinese hamster ovary (CHO) cells. The binding was abrogated by enzymatic removal of cell surface heparan sulfate proteoglycans by heparinase and heparitinase but not by treatment with condroitinase ABC. In addition, binding of the gB-Fc fusion protein to CHO cells was severely impaired in the presence of soluble heparin, as well as when heparan sulfate-deficient mutant CHO cells were used. Consistent with these findings, soluble heparin was found to block HHV-7 infection and syncytium formation in the SupT1 cell line. Although the CD4 antigen is a critical component of the receptor for the T-lymphotropic HHV-7, these findings suggest that heparin-like molecules also play an important role in HHV-7-cell surface interactions required for infection and that gB represents one of the HHV-7 envelope proteins involved in the adsorption of virus-to-cell surface proteoglycans.

HIV-1 attachment to host cells is generally considered to take place via high-affinity binding between CD4 and gp120. However, the binding of virion-associated gp120 to cellular CD4 is often weak, and most cell types that are permissive for HIV-1 infection express little CD4. Thus, other interactions between the virion and the cell surface could dominate the attachment process. It has been shown that host cell cyclophilin is incorporated into the viral particle at a rate of 200 molecules/particle, and cyclophilin binding via a basic heparin-like domain to HS mediates HIV-1 attachment and infectivity (Saphire A C et al, 1999 EMBO J. 18, 6771-6785).

Attachment of human cytomegalovirus (CMV) at the cell surface is rapid and efficient in permissive as well as non-permissive cell types, suggesting that cellular receptors for CMV are widely distributed. Addition of exogenous heparin or the treatment of cells with heparinase blocks viral attachment and implicates the proteoglycan heparan sulfate in the initial interaction between virus and cell. For human CMV it was shown that the envelope glycoprotein B (gB) is an important mediator of virus entry that works, at least in part, via heparin sulfate binding (Boyle K A and Compton T 1998, J Virol. 72, 1826-1833).

While some clinical benefit in ameliorating the sequelae of viral infection has been achieved by treatment with nucleoside analogues and interferons, therapy with both types of compounds can involve significant side effects. Patients treated with acyclovir, for example, may exhibit local inflammation at sites where the drug is administered, renal dysfunction, and encephalopathic changes. Experience in the use of vidarabine has revealed neurologic toxicity. Patients treated with interferon may exhibit fever, fatigue, anorexia, weight loss, nausea and vomiting, bone marrow suppression, pain at injection sites, lymphadenopathy, and mild hair loss. There thus exists a need in the art for additional products useful in preventing or treating viral infection.

SciFinder Scholar database lists 5 derivatives (as of Mar. 10, 2005) of thiazolideneethanesulfonic acid of the general Formula Ib described herein below, but no utility or chemical synthesis data is described.

SciFinder Scholar database lists 4288 derivatives (as of Mar. 10, 2005) of 3,5-disubstituted 2-thioxo-4-thiazolidinone of general Formula Ia described herein below, but no utility or chemical synthesis data is described.

Nowhere in the background art is it taught or suggested that compounds of the general formula I herein below have beneficial pharmaceutical activities.

### SUMMARY OF THE INVENTION

The present invention is directed to methods for the screening, identification and use of small organic molecules that modulate interactions and signaling events mediated by glycosaminoglycans (GAGs), particularly adhesion events between GAGs and viruses and, more specifically, between GAGs and specific GAG-binding viral proteins (GBVPs).

It is an object of some aspects of the present invention to provide pharmaceutical compositions comprising small organic compounds for medical and diagnostic use, wherein the small organic compounds are inhibitors of the interactions between GAGs and viruses and, more specifically, between heparan sulfate and specific GBVPs. Accordingly, these compositions are useful as inhibitors of virus attachment and entry. In addition, the compositions interact directly with HS-GAGs and are therefore useful as inhibitors of any HS-GAG mediated processes and conditions.

According to one aspect, the present invention provides a method of screening for small organic molecules that directly inhibit the interaction of GAGs with virus proteins, the method comprising the steps of:

(a) contacting a glycosaminoglycan (GAG) with a GAG-binding viral protein (GBVP) in the presence of at least one candidate compound; and

(b) measuring the amount of GAG bound to GBVP or the amount of GBVP bound to GAG, wherein a significant decrease in GAG-GBVP binding as compared to GAG-GBVP binding not in the presence of the candidate compound, identifies said compound as an inhibitor of GAG-GBVP interaction.

According to one embodiment, the GAG is immobilized before it is contacted with a GBVP.

According to another embodiment, the GBVP is immobilized before it is contacted with a GAG.

According to yet another embodiment, the GAG or the GBVP is tagged or labeled before measuring the GAG-GBVP binding. Tagging may be performed by use of a dye, a fluorescent dye, a chemiluminescent agent or a radioactive agent. Tagging of GBVP may be by an antibody directed to the specific GBVP or to a protein fused to the GBVP.

According to one embodiment, the small organic molecules screened by the methods of the present invention interact with GAGs selected from the group consisting of heparan sulfate (HS-GAG), heparin, chondroitin sulfate, dermatan sulfate, keratan sulfate and derivatives and fragments thereof.

According to one currently preferred embodiment, the glycosaminoglycans are HS-GAG or heparin or derivatives and oligosaccharide fragments thereof.

According to another embodiment, the small compounds screened by the methods of the present invention interact with proteoglycan containing GAG, particularly heparan sulfate proteoglycan (HS-PG).

According to one embodiment, the small organic molecules screened by the methods of the present invention inhibit the interaction of GAGs with GAG specific GBVPs, particularly human cytomegalovirus (CMV) envelope glycoprotein B.

According to one currently preferred embodiment, the small compounds screened by the methods of the present invention inhibit the interaction of GAGs with CMV envelope glycopotein B, particularly, the interaction of the GAG with the heparin binding domains of CMV envelope glycopotein B.

According to some other aspects, the present invention provides a pharmaceutical composition comprising a pharmaceutically acceptable diluent or carrier and as active ingredient an inhibitor compound identified by a screening method comprising the steps of:

(a) contacting a GAG with a GBVP in the presence of at least one candidate compound;

(b) measuring the amount of GAG bound to GBVP or the amount of GBVP bound to GAG, wherein a significant decrease in GAG-GBVP binding as compared to GAG-GBVP binding not in the presence of the candidate compound, identifies said compound as an inhibitor of GAG-GBVP interaction,

According to one embodiment, the pharmaceutical composition comprises an inhibitor compound that inhibits GAG-GBVP binding by interacting with GAGs selected from the group consisting of heparan sulfate (HS-GAG), heparin, chondroitin sulfate, dermatan sulfate, keratan sulfate and derivatives and fragments thereof.

According to one currently preferred embodiment, the pharmaceutical composition comprises an inhibitor compound that inhibits GAG-GBVP binding by interacting with HS-GAG or heparin or derivatives and oligosaccharide fragments thereof.

According to one currently preferred embodiment, the pharmaceutical composition comprises an inhibitor compound that inhibits the interaction of GAGs with CMV envelope glycoprotein B.

According to yet some other aspects, the present invention provides methods for modulation of virus attachment and entry in vivo mediated by interactions of GAGs and specific GBVPs.

According to one embodiment, the present invention provides a method for inhibiting virus attachment and entry in vitro comprising the step of exposing the cells to a small organic molecule that interacts directly with at least one GAG in an amount sufficient for preventing the interactions of the GAG with at least one specific GBVP.

According to another embodiment, the present invention provides a method for inhibiting virus attachment and entry in vivo comprising the step of administering a small organic molecule that interacts directly with at least one GAG in an amount sufficient for preventing the interactions of the GAG with at least one specific GBVP.

According to one embodiment, virus attachment and entry is inhibited by the interaction of the small compounds identified by the methods of the present invention with GAGs selected from the group consisting of heparan sulfate (HS-GAG), heparin, chondroitin sulfate, dermatan sulfate, keratan sulfate and derivatives, and fragments thereof.

According to one currently preferred embodiment, virus attachment and entry is inhibited by the interaction of the small organic molecules identified by the methods of the present invention with HS-GAG or heparin or derivatives and oligosaccharide fragments thereof.

According to yet another embodiment, virus attachment and entry is inhibited by the interaction of the small organic molecule identified by the methods of the present invention with proteoglycan containing GAG, preferably HS-PG.

According to one currently preferred embodiment, the small compounds identified by the methods of the present invention inhibit the interaction of GAGs with CMV envelope glycoprotein B.

According to a further aspect, the present invention provides a method for the treatment or prevention of disorders related to virus attachment and entry comprising the step of administering to a subject in need thereof a therapeutically effective amount of a small organic molecule identified by the methods of the present invention that directly inhibits the interaction of GAGs with GAG specific GBVPs, preventing virus attachment and entry mediated by the GAG.

According to one embodiment, the small organic molecule for the treatment or prevention of a disorder related to virus attachment and entry is identified by the screening method comprising the steps of:

(a) contacting a GAG with an GBVP in the presence of at least one candidate compound;

(b) measuring the amount of GAG bound to GBVP or the amount of GBVP bound to GAG, wherein a significant decrease in GAG-GBVP binding as compared to GAG-GBVP binding not in the presence of the candidate compound, identifies said compound as an inhibitor of GAG-GBVP interaction.

According to one embodiment, the disorder related to virus attachment and entry is CMV infection or HIV infection.

According to another embodiment, the small organic molecules of the present invention are administered for treating or preventing a viral disorder, condition or process exemplified by, but not restricted to, infections caused by hepatitis B virus (HBV), hepatitis C virus (HCV), HIV/AIDS, herpes HSV-1, HSV-2, and HSV-7 viruses, Cytomegalovirus (CMV), Respiratory syncytial virus (RSV), Varicella Zoster Virus (VZV), Influenza Virus, Rhinovirus, Epstein-Barr Virus, Human Papilloma Virus (HPV) and Dengue Virus.

According to yet another embodiment, the small organic molecules of the present invention are administered for treating or preventing other non-viral infectious diseases which involve adhesion processes and cell entry, including, but not limited to, bacterial infection and malaria.

According to one aspect, the present invention provides a pharmaceutical composition comprising a pharmaceutically acceptable diluent or carrier and an active ingredient of the general formula I:

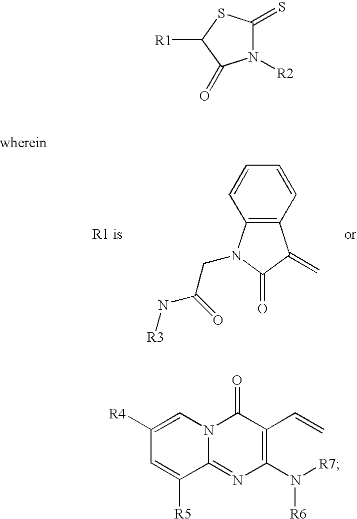

R2 is C₁-C₆ alkyl unsubstituted or substituted by a radical selected from the group consisting of —SO₃H, C₁-C₆ alkoxy, phenyl, 4-(C₁-C₆)alkylphenyl, 4-(C₁-C₆)alkoxyphenyl, 2-furyl, tetrahydro-2-furyl, or 1,3-benzodioxol-5-yl, or R₅ is cycloalkyl or C₂-C₆ alkenyl;

R3 is phenyl substituted by at least one radical selected from the group consisting of C₁-C₆ alkyl, hydroxy(C₁-C₆)alkyl, C₁-C₆ alkoxy, cyano, halogen, trifluoromethyl, cycloalkyl, aralkyl, aryl, substituted aryl, and heterocyclyl;

R4 and R5 each is hydrogen or C₁-C₆ alkyl;

R6 and R7 each is selected from the group consisting of C₁-C₆ alkyl, C₁-C₆ alkyl substituted by piperidinyl, 4-morpholinyl, piperazinyl, 4-(C₁-C₆)alkyl-piperazinyl, 4-arylpiperazinyl, 4-aralkylpiperazinyl, or imidazolyl; C₃-C₇ cycloalkyl, C₆-C₁₀aryl, C₇-C₁₆ aralkyl, and C₇-C₁₆ aralkyl, or R₃ and R₄ together with the nitrogen atom to which they are attached form a 5 to 7 membered saturated heterocyclic ring containing one or two heteroatoms and optionally substituted at the additional nitrogen atom by C₁-C₆ alkyl optionally substituted by halogen, hydroxyl, C₁-C₆ alkoxy or phenyl, or C₂-C₇ alkoxycarbonyl, and pharmaceutically acceptable salts thereof.

In one embodiment, the pharmaceutical compositions of the present invention comprises a compound of the general formula Ia:

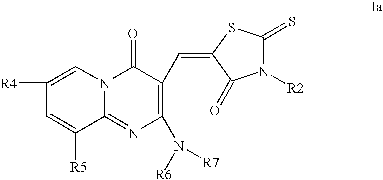

wherein:

R2 is C₁-C₆ alkyl unsubstituted or substituted by C₁-C₆ alkoxy, phenyl, 4-(C₁-C₆)alkylphenyl, 4-(C₁-C₆)alkoxyphenyl, 2-furyl, tetrahydro-2-furyl, or 1,3-benzodioxinyl, or R₅ is cycloalkyl or alkenyl; R4 and R5 each is hydrogen or C₁-C₆ alkyl;

R6 and R7 each is selected from the group consisting of C₁-C₆ alkyl, C₁-C₆ alkyl substituted by piperidinyl, 4-morpholinyl, piperazinyl, 4-(C₁-C₆)alkyl-piperazinyl, 4-arylpiperazinyl, 4-aralkylpiperazinyl, or imidazolyl; C₃-C₇ cycloalkyl, C₆-C₁₀ aryl, C₇-C₁₆ aralkyl, and C₇-C₁₆ aralkyl, or R₃ and R₄ together with the nitrogen atom to which they are attached form a 5 to 7 membered saturated heterocyclic ring containing one or two heteroatoms and optionally substituted at the additional nitrogen atom by C₁-C₆ alkyl optionally substituted by halogen, hydroxyl, C₁-C₆ alkoxy or phenyl, or C₂-C₇ alkoxycarbonyl;

and pharmaceutically acceptable salts thereof.

According to certain embodiments, the compounds of formula Ia include the following compounds:

4H-Pyrido[1,2-a]pyrimidin-4-one, 3-[[3-[(2-methylpropyl)]-4-oxo-2-thioxo-5-thiazolidinylidene]methyl]-2-[4-(2-hydroxyethyl)-1-piperazinyl]- (Compound 1)

4H-Pyrido[1,2-a]pyrimidin-4-one, 3-[[3-(phenylethyl)-4-oxo-2-thioxo-5-thiazolidinylidene]methyl]-2-[[2-(4-morpholinyl)ethyl]amino]-9-methyl-(Compound 2)

4H-Pyrido[1,2-a]pyrimidin-4-one, 3-[(3-pentyl-4-oxo-2-thioxo-5-thiazolidinylidene)methyl]-2-(4-methyl-1-piperazinyl)- (Compound 3)

4H-Pyrido[1,2-a]pyrimidin-4-one, 3-[[3-(phenylmethyl)-)-4-oxo-2-thioxo-5-thiazolidinylidene]methyl]-2-(4-methyl-1-piperazinyl)-(Compound 4)

4H-Pyrido[1,2-a]pyrimidin-4-one, 3-[(3-phenylmethyl-4-oxo-2-thioxo-5-thiazolidinylidene)methyl]-2-(4-methyl-1-piperazinyl)-7-methyl- (Compound 5)

4H-Pyrido[1,2-a]pyrimidin-4-one, 3-[[3-[(4-methoxyphenyl)methyl]-4-oxo-2-thioxo-5-thiazolidinylidene]methyl]-2-(4-methyl-1-piperazinyl)- (Compound 6)

4H-Pyrido[1,2-a]pyrimidin-4-one, 3-[(3-butyl-4-oxo-2-thioxo-5-thiazol-idinylidene)methyl]-9-methyl-2-(4-methyl-1-piperazinyl)- (Compound 10)

4H-Pyrido[1,2-a]pyrimidin-4-one, 3-[(3-phenylmethyl-4-oxo-2-thioxo-5-thiazolidinylidene)methyl]-2-[[3-(1H-imidazol-1-yl)propyl]amino]- (Compound 25)

4H-Pyrido[1,2-a]pyrimidin-4-one, 3-[[3-(phenylmethyl)-4-oxo-2-thioxo-5-thiazolidinylidene]methyl]-2-[[2-(4-morpholinyl)ethyl]amino]-9-methyl- (4428-0223, Compound 26).

According to another embodiment, the pharmaceutical compositions of the present invention comprises a compound of the general formula Ib:

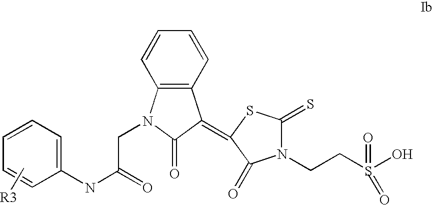

wherein:

R3 is C₁-C₁₀ alkyl, hydroxy(C₁-C₁₀)alkyl, C₁-C₆ alkoxy, cyano, halogen, trifluoromethyl, cycloalkyl, aralkyl, aryl, substituted aryl, and heterocyclyl; and pharmaceutically acceptable salts thereof.

According to one embodiment, R3 is methyl, ethyl, or hydroxyethyl. According to another embodiment, R3 is halogen, cyano, 3,4-dicyano, methoxy, 4,5-dimethoxy, or 3-trifluoromethyl.

According to certain embodiments, the compounds of formula Ib include the compounds:

5-[1,2-dihydro-2-oxo-1-[2-oxo-2-[[3-(trifluoromethyl)phenyl]amino]ethyl]-3H-indol-3-ylidene]-4-oxo-2-thioxo-3-thiazolidineethanesulfonic acid [Compound 11]; and

5-[1,2-dihydro-2-oxo-1-[2-oxo-2-[3-(cyanophenyl)amino]ethyl]-3H-indol-3-ylidene]-4-oxo-2-thioxo-3-thiazolidineethanesulfonic acid.

According to another aspect, the present invention relates to the use of a compound of the general formula I for the preparation of a pharmaceutical composition, particularly for use as antiviral composition.

According to some aspects, the present invention provides a method for the treatment or prevention of diseases and disorders related to virus attachment and entry mediated by GAG-GBVP interactions, comprising the step of administering to a subject in need thereof a pharmaceutical composition comprising a therapeutically effective amount of at least one compound of the general formula I. In one embodiment, the composition comprises a compound of the general formula Ia. In another embodiment, the composition comprises a compound of the general formula Ib.

According to some aspects the present invention provides a method for the treatment or prevention of non-viral infectious which involve adhesion processes and cell entry, including, but not limited to, bacterial infection and malaria, comprising the step of administering to a subject in need thereof a pharmaceutical composition comprising a therapeutically effective amount of at least one compound of the general formula I. In one embodiment, the composition comprises a compound of the general formula Ia. In another embodiment, the composition comprises a compound of the general formula Ib.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1 shows CMV glycoprotein B binding to immobilized heparin.

FIG. 2 demonstrates inhibition of CMV glycoprotein B binding to immobilized heparin by soluble heparin.

### DETAILED DESCRIPTION OF THE INVENTION

It is an object of the present invention to provide methods for screening and identifying compounds capable of inhibiting interaction between glycosaminoglycans (GAGs) and specific GAG-binding viral proteins (GBVPs).

It is another object of the present invention to provide methods for screening and identifying compounds capable of direct inhibition of GAG-mediated virus attachment and entry into cells.

It is another object of some aspects of the present invention to provide pharmaceutical compositions comprising small organic compounds for medical and diagnostic use, wherein the small organic compounds are inhibitors of the interactions between GAGs and viruses and, more specifically, between heparan sulfate and specific GBVPs. Accordingly, these compositions are useful as inhibitors of virus attachment and entry. In addition, the compositions interact directly with HS-GAGs and are therefore useful as inhibitors of any HS-GAG mediated processes and conditions.

It is yet another object of the present invention to provide methods for the treatment of diseases or disorders associated with virus attachment and entry into cells mediated by the interactions between GAGs and specific GBVPs.

Definitions

In accordance with the present invention and as used herein, the following terms are defined with the following meanings, unless explicitly stated otherwise.

The term “Compound” refers to small organic molecule having a molecular weight less than 1500 Daltons and preferably between 300 to 1200 Daltons.

The term “HS-GAG” refers to heparan sulfate glycosaminoglycan. It includes fragments of heparan sulfate such as those that may be produced chemically, enzymatically or during purification. It includes the HS-GAG chains of proteoglycans such as heparan sulfate proteoglycans. HS-GAG may be free or attached to a linker, support, cell or protein, or otherwise chemically or enzymatically modified. HS-GAGs may be crude or purified from organs, tissues or cells.

The term “GAG” refers to glycosaminoglycans, including heparan sulfate (that is referred to in the art also as HS-GAG), heparin, chondroitin sulfate, dermatan sulfate and keratan sulfate. It includes the GAG chains of proteoglycans such as heparan sulfate proteoglycan or chondroitin sulfate proteoglycan.

“HS-PG” or “HSPG” refers to heparan sulfate proteoglycans.

“Heparin” is polysulfated polysaccharide, with no protein associated with it. As used herein, heparin refers to heparin prepared from different organs or species such as porcine intestinal mucosa heparin. It includes low molecular weight heparins, such as commercially available Fraxiparin, and other heparin derivatives, prepared or modified by chemical or enzymatic reaction.

“Heparin derivatives” consist of products derived from heparin, made by one or more chemical or enzymatic modifications. The modifications are designed to change the activity of relevant groups of the molecules.

“Heparin derived oligosaccharides” are products made from heparin by controlled cleavage and subsequent purification.

“Heparan derivatives” consist of products derived from heparan sulfate, made by one or more chemical or enzymatic modifications. The modifications are designed to change the activity of relevant groups of the molecules.

“Heparan derived oligosaccharides” are products made from heparan sulfate by controlled cleavage and subsequent purification.

The term “Specific GBVP” means a specific viral protein adhesion molecule and refers to a GAG-binding protein molecule involved in mediating virus attachment and virus-cell interaction and having a heparin-binding domain, for example CMV envelope glycoprotein B and the like. It includes mutant proteins, protein domains, peptide fragments and the like, that retain the GAG binding domain (heparin-binding domain).

The term “Inhibitor compound” refers to a small organic molecule that inhibits the interaction (binding) between two molecules: (1) a GAG, exemplified by, but not restricted to heparin or HS-GAG and (2) an GBVP, exemplified by, but not restricted to CMV envelope glycoprotein B.

The term “Synthetic chemical compound collection” or “Compound collection” refers to a collection of random and semi-random synthetic molecules wherein each member of such collection or library is produced by chemical or enzymatic synthesis.

The term “Treatment” or “Treating” is intended to include the administration of the compound of the invention to a subject for purposes which may include prophylaxis, amelioration, prevention or cure of disorders mediated by virus attachment and infection events. Such treatment need not necessarily completely ameliorate the viral infection or other responses related to the specific disorder. Further, such treatment may be used in conjunction with other traditional treatments for reducing the disease or disorder condition known to those of skill in the art.

The methods of the invention may be provided as a “Preventive” treatment before detection of, for example, a viral infection, so as to prevent the disorder from developing in patients at high risk for the same, such as, for example, transplant patients.

As used through this specification, the singular forms “a”, “an” and “the” include the plural unless the context clearly dictates otherwise. Thus, for example, reference to “a compound” includes mixtures of such compounds, reference to “a P-selectin”, or “an L-selectin” includes reference to respective mixtures of such molecules, reference to “the formulation” or “the method” includes one or more formulations, methods and/or steps of the type described herein and/or which will become apparent to those persons skilled in the art upon reading this disclosure.

Methods for Compound Screening and Drug Discovery

Currently, attempts at modulating GAG interactions with GAG specific GBVPs are indirect, targeting the heparin-binding domains associated with specific GBVPs by using GAG-mimetics such as heparins, derivatives and other sulfated GAG mimetics.

The present invention provides a method for screening and identifying compounds for drug development, disclosing GAGs, specifically HS-GAGs, as novel molecular targets for such screening. The direct targeting of GAGs as described herein is of critical importance, since modem drug discovery requires precise knowledge of the molecular nature of the drug binding site for efficient drug screening and chemical optimization.

According to one aspect, the present invention provides a method of screening for small compounds that directly inhibit the interaction of GAGs with specific GBVPs, the method comprising the steps of:

(a) contacting a GAG with a GBVP in the presence of at least one candidate compound; and

(b) measuring the amount of GAG bound to GBVP or the amount of GBVP bound to GAG, wherein a significant decrease in GAG-GBVP binding as compared to GAG-GBVP binding not in the presence of the candidate compound, identifies said compound as an inhibitor of GAG-GBVP interaction.

The compound screening methods for identification of inhibitor compounds may be used in various modifications, which are well known to one skilled in the art. Assays can be classified as either direct binding assays or inhibition assays. The GAG molecule may be immobilized, or GBVP may be immobilized, or both GAG and GBVP may be present in solution. Detection may focus either on GAG or on GBVP, for instance, by using antibodies, biotin-streptavidin, radiolabeling, and fluorescent label. Detection methods may also differ, for example, spectrophotometry, chemoluminiscence, fluorescence, and radioactive detection may be employed. Immobilized GAGs may be used coated on plates or coupled to beads. GAGs may be linked to a carrier such as a protein, using different chemical methods. Alternatively, the GBVPs may be immobilized, for instance, by coating plates or coupling to beads. GBVPs may be used as fusion proteins or domains containing the GAG-binding domain. Another useful approach may be to use as a source of GAG a whole cell such as a fibroblast cell. This is particularly relevant for identifying inhibitor compounds that prevent adhesion to such fibroblast cells.

According to one embodiment, compounds for screening may be produced by synthetic chemistry or may be natural compounds, individual or in mixtures, pre-selected by an algorithm, compressed libraries and the like. A preferred method of screening is known as high-throughput screening (HTS), in which thousands of compounds are screened with the aid of robotics.

According to one currently preferred embodiment, compound screening according to the method of the present invention is used as iterative screening in conjunction with chemical optimization via synthetic chemistry.

According to one embodiment, the small organic molecules screened by the methods of the present invention interact with GAGs selected from the group consisting of heparan sulfate (HS-GAG), heparin, chondroitin sulfate, dermatan sulfate, keratan sulfate, and derivatives and fragments thereof.

According to one currently preferred embodiment, the glycosaminoglycans are HS-GAG or heparin or derivatives and oligosaccharide fragments thereof. GAGs may be crude or purified from an organ, tissue or cell. Such HS-GAGs may be commercially available, or purified from source of interest such as human liver, human brain, endothelial cells and the like. The HS-GAGs may be also chemically or enzymatically modified, or produced synthetically.

According to another embodiment, the small compounds screened by the methods of the present invention interact with proteoglycan containing GAG, particularly heparan sulfate proteoglycan (HS-PG). Proteoglycans having HS-GAG chains may be purified from an organ, tissue, cell or tumor. Examples for such HS-PGs are syndecan or aggrin. Proteoglycans having other GAG chains, such as versican, may be also used.

According to one currently preferred embodiment, the small compounds screened by the methods of the present invention inhibit the interaction of GAGs with CMV envelope glycoprotein B, namely the interaction of the GAG with the heparin binding domain of CMV envelope glycoprotein B.

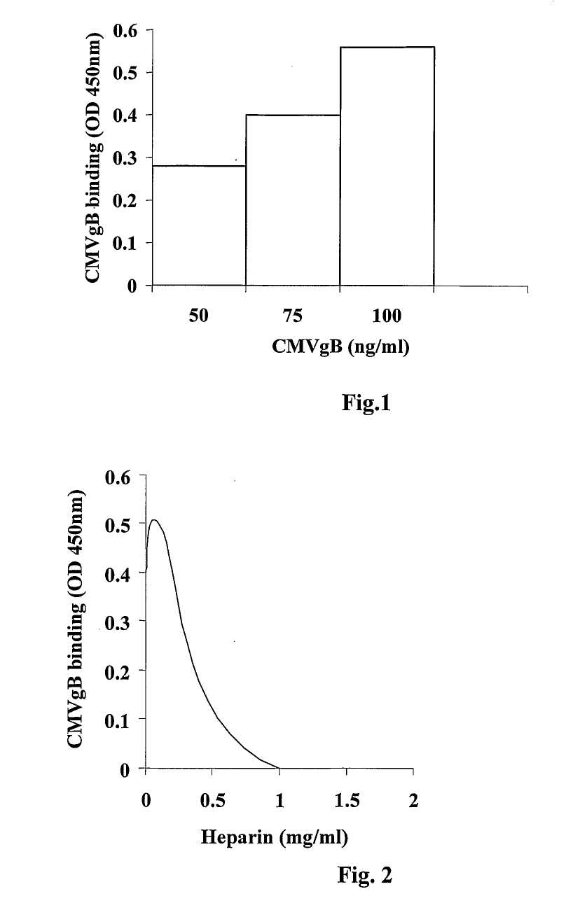

The present invention discloses for the first time the use of CMV envelope glycoprotein B for compound screening and for direct targeting of GAG binding sites. The screening method of the present invention is based on an ELISA assay for CMV envelope glycoprotein B interaction with heparin on 96-well plates, suitable for screening compound collections, newly developed by the inventors of the present invention. The assay measures binding of CMV envelope glycoprotein B to immobilized heparin. The amount of bound CMV envelope glycoprotein B is determined by an ELISA assay using a monoclonal antibody conjugated to horseradish peroxidase. FIG. 1 shows CMV envelope glycoprotein B binding to heparin. Soluble heparin was found according to the present invention to inhibit CMV envelope glycoprotein B binding to immobilized heparin (FIG. 2). This method can be used with other specific GBVPs such as HSV-1 glycoproteins B, C, and D, HHV-7 glycoprotein B, and HCV envelope protein. Additionally, other GAGs are capable of replacing heparin in this kind of assay. In particular, in place of heparin one may immobilize a different HS-GAG such as purified HS-GAG from an organ, tissue or cell of interest. HS-GAGs may be immobilized by methods similar for immobilization of heparin, or by other means known in the art.

Preferably, when using this kind of assay for compound screening, one may use a particular GAG or PG from a target tissue, such as endothelial cell HS-GAG, kidney purified HS-GAG or HS-PG, and the like. The reason is that molecular diversity of HS-GAGs is regulated in a tissue and cell-specific manner and different HS-GAGs have different binding sites for GAG specific GBVPs.

The present invention demonstrates for the first time that this kind of GAG-viral protein interaction assay is suitable for screening collections of compounds and for discovery of novel drugs. As described herein below, the CMV envelope glycoprotein B assay was used to screen a collection of several thousand compounds on 96-well plates. For this purpose, the compounds in individual wells were co-incubated with CMV envelope glycoprotein B on plates containing immobilized heparin. Following completion of assay and color development, percentage of inhibition obtained for each compound was determined. Positive and negative controls were included on every plate; heparin was used as positive control. Compounds that inhibited at least 30% of the signal were scored as hits and selected for further analysis. Examples of inhibitor compounds are given in Example 4, Table 1.

According to one embodiment of the present invention, the inhibitor compounds identified by the methods of the present invention directly interact with GAGs and inhibit their interaction with specific GBVPs.

In principle, the inhibitor compounds can inhibit CMV envelope glycoprotein B-heparin interaction either (i) by direct binding to heparin and thus preventing its interaction with CMV envelope glycoprotein B or (ii) by direct binding to CMV envelope glycoprotein B and subsequently preventing its interaction with heparin. A third theoretical possibility is that the compound binds to both heparin and CMV envelope glycoprotein B.

Compounds found to be suitable for further development and chemical optimization may be further subjected to a second screening, identifying those that directly bind to heparin. Individual compounds are incubated with immobilized heparin in the absence of CMV envelope glycoprotein B. After washing of the plates to remove all unbound compound, CMV envelope glycoprotein B is added. At this time, in separate wells, CMV envelope glycoprotein B is co-incubated with the test compounds and the standard assay protocol is followed. Test compounds which bind directly and irreversibly to heparin are identified by comparing the results of pre-incubation versus co-incubation experiments.

As exemplified for the first time by the present invention, structurally diverse compounds are capable of inhibiting GAG interactions with GBVPs. Such inhibitor compounds may have therapeutic implications and may be useful for a variety of disorders, since GAGs and GBVPs have many biological roles and have been implicated in a multitude of disorders.

Methods for Modulating Virus Attachment and Entry into Cells

According to another aspect, the present invention provides methods for modulation of virus attachment and entry in vivo or in vitro mediated by interactions of GAGs and GAG specific GBVPs.

According to one embodiment, the present invention provides a method for inhibiting virus attachment and entry in vitro comprising the step of exposing the cells to a small organic molecule that interacts directly with at least one GAG in an amount sufficient for preventing the interactions of the GAG with at least one specific GBVP.

According to another embodiment, the present invention provides a method for inhibiting virus attachment and entry in vivo comprising the step of administering a small organic molecule that interacts directly with at least one GAG in an amount sufficient for preventing the interactions of the GAG with at least one GBVP.

According to one embodiment, virus attachment and entry is inhibited by the interaction of the small compounds identified by the methods of the present invention with GAGs selected from the group consisting of heparan sulfate (HS-GAG), heparin, chondroitin sulfate, dermatan sulfate, keratan sulfate and derivatives and fragments thereof.

According to one currently preferred embodiment, virus attachment and entry is inhibited by the interaction of the small organic molecules identified by the methods of the present invention with HS-GAG or heparin.

According to yet another embodiment, virus attachment and entry is inhibited by the interaction of the small organic molecule identified by the methods of the present invention with proteoglycan containing GAG, preferably HS-PG.

According to one currently preferred embodiment, the small compounds identified by the methods of the present invention inhibit the interaction of GAGs with CMV envelope glycoprotein B.

Methods for Treatment of Disorders Related to Virus Attachment and Entry into Cells.

According to yet another aspect, the present invention provides a method for the treatment or prevention of disorders related to virus attachment and entry comprising the step of administering to a subject in need thereof a therapeutically effective amount of a small organic molecule identified by the methods of the present invention, that directly inhibits the interaction of GAGs with a GBVPs, preventing virus attachment and entry mediated by the GAG.

Blocking virus attachment and entry has proven to be highly effective in the treatment of number of viral diseases and disorders including HIV.

According to one embodiment, the small organic molecule for the treatment or prevention of a disorder related to virus attachment and entry is identified by the screening method comprising the steps of:

(a) contacting a GAG with an GBVP in the presence of at least one candidate compound;

(b) measuring the amount of GAG bound to GBVP or the amount of GBVP bound to GAG, wherein a significant decrease in GAG-GBVP binding as compared to GAG-GBVP binding not in the presence of the candidate compound, identifies said compound as an inhibitor of GAG-GBVP interaction.

According to another embodiment the GAGs of the inhibited GAG-GBVP interactions are selected from the group consisting of heparan sulfate (HS-GAG), heparin, chondroitin sulfate, dermatan sulfate, keratan sulfate, and derivatives and fragments thereof.

According to one currently preferred embodiment, the GAGs of the inhibited GAG-GBVP interactions are selected from the group consisting of HS-GAG and heparin.

According to yet another embodiment the GBVPs of the inhibited GAG-GBVP interactions are selected from the group consisting of CMV envelope glycoprotein B, HSV-1 glycoprotein B, HSV-1 glycoprotein C and HCV envelope protein.

According to one currently preferred embodiment, the GBVP that inhibits GAG-GBVP interactions consists of the CMV envelope glycoprotein B.

Virus attachment is the first event in the pathological cascade of the viral infection. Viral envelope proteins, those which contact and mediate fusion with viral host cells, are therefore potential targets for anti-viral therapies (Faulkner L et al 2003, Vaccine 21:932-9).

The present invention discloses methods of screening for small organic molecules capable of inhibiting GAG interaction with GBVPs; the present invention further shows that such inhibitor compounds are useful as inhibitors of virus-cell adhesion processes and, moreover, are useful for the prevention or treatment of diseases associated with virus attachment, entry and infection.

According to one embodiment, the small organic molecules of the present invention are administered for treating or preventing a viral disorder, condition or process exemplified by, but not restricted to AIDS, and viral infection caused by HIV, CMV, RSV, HSV, VZV.

According to one embodiment, the inhibitor compounds inhibit virus attachment, entry or infection.

Human herpes viruses (HHVs) are human pathogens which cause a variety of disease states including cold sores, eye and genital infections, life-threatening neonatal infections, and encephalitis.

Dengue virus is a human pathogen that has reemerged as an increasingly important public health threat. Heparin, highly sulfated heparan sulfate, and the polysulfonate pharmaceutical Suramin effectively prevented dengue virus infection of target cells, indicating that the envelope protein-target cell receptor interaction is a critical determinant of infectivity. The dengue envelope protein sequence includes two putative GAG-binding motifs at the carboxy terminus; the first could be structurally modeled and formed an unusual extended binding surface of basic amino acids. Similar motifs were also identified in the envelope proteins of other flaviviridae. Developing pharmaceuticals that inhibit target cell binding may be an effective strategy for treating flavivirus infections.

The cytomegaloviruses (CMVs) are a distinct, widely distributed sub-group of herpes viruses. In most areas of the world, human CMV spreads at an early age and affects a large majority of the population. The importance of CMV as a pathogen has arisen with the increase in organ allografting and immunosuppressive post-transplant therapies and the increase in acquired immunodeficiency syndrome (AIDS). These conditions predispose individuals to a primary CMV infection or to reactivation of latent infection, which may lead to fulminant, life-threatening disease. The virion carries two prominent herpes-virus-conserved glycoprotein complexes. One is composed of covalently linked, proteolytically processed, dimers of glycoprotein B (gB), which plays a critical role in viral entry. gB is the major HS proteoglycan-binding glycoprotein (Compton T 1993, Virol 193:84-841). The heparin binding properties of a synthetic peptide deduced from the sequence of human CMV gB were investigated (Silvestri M E and Sundquist V A 2001, Scan. J Immunol. 53:282-9). The peptide bound heparin in vitro and bound to human cells in a manner suggesting an interaction with extracellular matrix. Binding of the peptide to human fibroblasts could be inhibited both by adding soluble heparin and by enzymatic pretreatment of the cells with heparinase. This evidence indicates that CMV gB binding to cell surface HS is a critical step in viral attachment and infection.

According to another embodiment, the small organic molecules of the present invention are administered for treating or preventing malaria. Severe Plasmodium falciparum malaria is characterized by excessive sequestration of infected and uninfected erythrocytes in the microvasculature of the infected organ. Roseting, the adhesion of P. falciparum-infected erthrocytes to uninfected erthroctyes, is a virulent parasite phenotype associated with the occurrence of severe malaria. The adhesion ligand, P. falciparum erthrocyte membrane protein 1 (PfEMP1), contains clusters of GAG-binding motifs (Chen Q et al 1998, J Exp. Med. 187:15-23). The adhesive interactions could be inhibited with HS or heparitinases. P. falciparum is another example of an infectious agent which has evolved a molecular mechanism to exploit cell surface GAGs to facilitate or effect cell entry.

According to another embodiment, the small organic molecules of the present invention are administered for treating or preventing bacterial infections. Present in the extracellular matrix and membranes of virtually all animal cells, GAGs are among the first host macromolecules encountered by infectious agents. Pathogenic bacteria exploit the GAGs to attach to target cells using bacterially expressed “adhesins” (Menozzi F D et al 2002, Mol. Microbiol. 43:1379-86). Some pathogens, such as Bordetella pertussis and Chlamydia trachomatis, may express more than one GAG-binding adhesins. Bacterial interactions with PGs may also facilitate cell invasion or system dissemination, as observed for Neisseria gonorrhoeae and Mycobacterium tuberculosis, respectively. A specific example of enhanced bacterial virulence through exploitation of host GAGs is the Lyme disease spirochaete (Parveen N et al 2003, Mol. Microbiol. 47:1433-44). The Lyme disease spirochaete, Borrelia burgdorferi, is transmitted to mammals by Ixodes ticks and can infect multiple tissues. Host cell attachment may be critical for tissue colonization and B. burgdorferi cultivated in vitro recognizes heparin and dermatan sulfate-related GAGs on the surface of mammalian cells. Host-adapted B. burgdorferi exhibited approximately three fold better binding to purified GAGs and those expressed on the surface of cultured endothelial cells. Three B. burgdorferi surface proteins, Bgp, DbpA and DbpB bind to GAGs and were shown to be present on the bacterial surface at higher levels after host adaptation.

Pharmaceutical Compositions

The pharmaceutical compositions described herein comprise compounds that were identified as inhibitor compounds according to the present invention. Certain compounds were discovered in the drug screening procedure described in Example 4 and Table 1. Other compounds were discovered as inhibitor compounds directly interacting with GAGs, see Example 5 and Table 2. Some other inhibitor compounds were discovered to inhibit virus infectivity in cell culture systems, see Examples 6 and 7.

According to one aspect, the present invention provides a pharmaceutical composition comprising as an active ingredient a compound of the general formula I hereinabove. In one embodiment, the composition comprises a compound of formula Ia. In another embodiment, the composition comprises a compound of formula Ib.

As defined herein, the term “C₁-C₆ alkyl” typically refers to a straight or branched alkyl radical having 1-6 carbon atoms and includes, for example, methyl, ethyl, n-propyl, isopropyl, n-butyl, isobutyl, tert-butyl, n-pentyl, isopentyl, 2,2-dimethylpropyl, n-hexyl, and the like. The term “C₂-C₆ alkenyl” refers to a straight or branched hydrocarbon radical having 2-6 carbon atoms and one or more double bonds, and includes for example vinyl, allyl, but-3-en-1-yl, pent-4-en-1-yl, hex-5-en-1-yl, and the like.

The term “C₁-C₆ alkoxy” typically refers to a straight or branched alkoxy radical having 1-6 carbon atoms and includes, for example, methoxy, ethoxy, n-propyloxy, isopropyloxy, n-butoxy, isobutoxy, tert-butoxy, n-pentyloxy, isopentyloxy, n-hexyloxy, and the like.

The term “cycloalkyl” refers to a saturated cycloalkyl radical having 3-8 carbon atoms and includes, for example, cyclopropyl, cyclobutyl, cyclopentyl, cyclohexyl, and the like.

The term “aryl” refers to a C₆-C₁₀ carbocyclic aryl radical such as phenyl or naphthyl. The term “aralkyl” refers to such an aryl radical covalently linked to a “C₁-C₆ alkyl” and includes benzyl, phenethyl, phenylpropyl. and the like. The aryl may be substituted by halogen, trifluoromethyl, C₁-C₆ alkyl, C₁-C₆ alkoxy, cyano.

The term “halogen” refers to fluoro, chloro, bromo and iodo.

The term “heterocyclyl” refers to a radical derived from a mono- or poly-cyclic ring containing one to three heteroatoms selected from the group consisting of N, O and S, with or without unsaturation or aromatic character. The term “heteroaryl” refers to such a mono- or poly-cyclic ring having aromatic character. Non-limiting examples of non-aromatic heterocyclyl include dihydrofuryl, tetrahydrofuryl, dihydrothienyl, pyrrolydinyl, pyrrolynyl, dihydropyridyl, piperidinyl, piperazinyl, morpholino,1,3-dioxanyl, and the like. A polycyclic ring may have the rings fused, as in quinoline or benzofuran, or unfused as in 4-phenylpyridine. Non-limiting examples of heteroaryl include pyrrolyl, furyl, thienyl, pyrazolyl, imidazolyl, oxazolyl, isoxazolyl thiazolyl, isothiazolyl, pyridyl, 1,3-benzodioxinyl, pyrazinyl, pyrimidinyl, 1,3,4-triazinyl, 1,2,3-triazinyl, 1,3,5-triazinyl, thiazinyl, quinolinyl, isoquinolinyl, benzofuryl, isobenzofuryl, indolyl, imidazo[1,2-a]pyridyl, pyrido[1,2-a]pyrimidinyl, benzimidazolyl, benzthiazolyl, benzoxazolyl and the like. It is to be understood that when a polycyclic heteroaromatic ring is substituted, the substitutions may be in any of the carbocyclic and/or heterocyclic rings.

When R6 and R7 together with the nitrogen atom to which they are attached form a 5-7 membered saturated heterocyclic ring containing one or two heteroatoms, the heteroatoms are selected from N, O and S, Examples of such rings include, without limitation, pyrrolidine, imidazolidine, oxazolidine, thiazolidine, piperidine, piperazine, morpholino and thiomorpholino. The substituent at the additional nitrogen atom may be C₁-C₆ alkyl, optionally substituted by halo, hydroxy, C₁-C₆ alkoxy or C₆-C₁₀ aryl, or C₂-C₇ alkoxycarbonyl.

Methods of Treatment of Viral Disorders

According to some aspects the present invention provides a method for the treatment or prevention of diseases and disorders related to virus attachment and entry mediated by GAG-GBVP interactions, comprising the step of administering to a subject in need thereof a pharmaceutical composition comprising a therapeutically effective amount of at least one compound of the general formula I.

The term “Pharmaceutically acceptable” means approved by a regulatory agency of the US federal or a state government or listed in the U.S. pharmacopeia or other generally recognized pharmacopeia for use in animals, and more particularly in humans.

The term “Carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents.

The pharmaceutical compositions of the invention can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, and magnesium carbonate. Examples of suitable pharmaceutical carriers are described in “Remington's Pharmaceutical Sciences” by E. W. Martin. Such compositions will contain a therapeutically effective amount of the compound, preferably in purified form, together with a suitable amount of carrier so as to provide the form for proper administration to the subject. The formulation should suit the mode of administration.

According to one embodiment, the pharmaceutical composition comprises an inhibitor compound that inhibits GAG-GBVP binding by interacting with GAGs selected from the group consisting of heparan sulfate (HS-GAG), heparin, chondroitin sulfate, dermatan sulfate, keratan sulfate and derivatives and fragments thereof.

According to one currently preferred embodiment, the pharmaceutical composition comprises an inhibitor compound that inhibits GAG-GBVP binding by interacting with HS-GAG or heparin or derivatives and oligosaccharide fragments thereof.

According to one currently preferred embodiment, the pharmaceutical composition comprises an inhibitor compound that inhibits the interaction of GAGs with CMV envelope glycoprotein B.

The following examples are put forth so as to provide those of ordinary skill in the art with a complete disclosure description of how to make the assays, the assay components, and carry out the assays of the invention and are not intended to limit the scope of what is regarded as the invention.

### EXAMPLES

### Example 1

An assay for CMV Envelope Glycoprotein B Binding to Immobilized Heparin that is Suitable for Screening of Compound Collections.

Porcine intestinal mucosa heparin conjugated to bovine serum albumin (Heparin-BSA; Sigma Cat. No. H0403) at 5 mg/ml in phosphate buffered saline (PBS; pH 6.5) was added to a 96-well polystyrene ELISA plate (NUNC Cat. No. 442404; 0.1 ml per well) and incubated over night at 4° C. Following the incubation the plate was washed consecutively, by immersion, with de-ionized water and PBS (pH 6.5). The ELISA plate was then blocked with BSA (ICN Cat. No. 160069, 3%, 200 μl per well) for 1 hour at room temperature. Following blocking, the plate was washed with de-ionized water then PBS (pH 6.5) plus Tween 20 (0.05%). CMV envelope glycoprotein B (Research Diagnostics, INC. Cat. No. RDI-RCMVAG-B) dissolved in PBS (supplemented with BSA (0.1%)) was added to the ELISA plate (100 μl per well) and incubated for 60 minutes at room temperature with shaking. Following incubation, the plate was washed with de-ionized water and with PBS (pH 6.5) plus Tween. Mouse anti-human cytomegalovirus gB antibody (Research Diagnostics, INC. Cat. No. RDI-CMVG Babm) diluted in PBS (supplemented with BSA (0.1%)), 1:2000, was added to the ELISA plate (100 μl per well) and incubated for 90 minutes at room temperature with shaking. Following the incubation, the plate was washed with de-ionized water and PBS (pH 6.5) plus Tween. Goat anti-mouse IgG (H&L) Peroxidase Conjugated antibody (Chemicon International, Inc. Cat. No. AP124P) diluted in PBS (supplemented with BSA (0.1%)), 1:1000, was added to the ELISA plate (100 μl per well) and incubated for 30 minutes at room temperature with shaking. Following the incubation, the plate was washed with de-ionized water and with PBS (pH 6.5) plus Tween. The peroxidase substrate chromogen, TMB (Dako Cat. No. S1599) was added (100 μl per well) to the ELISA plate and incubated at room temperature. After 15 minutes ELISA Stop Solution (hydrochloric acid 1N, sulfuric acid 3N) was added (200 μl per well) to stop the peroxidase catalyzed colorimetric reaction. The Optical Density (OD) of the samples was measured at 450 nm using an ELISA plate reader (Dynatech MR5000). A dose response histogram is shown in FIG. 1.

### Example 2

Inhibition of Human CMV Envelope Glycoprotein B Binding to Heparin by Soluble Heparin.

Porcine intestinal mucosa heparin conjugated to Bovine Serum Albumin (Heparin-BSA; Sigma Cat. No. H0403) at 5 mg/ml in Phosphate Buffered Saline (PBS; pH 6.5) was added to a 96-well polystyrene ELISA plate (NLNC Cat. No. 442404; 0.1 ml per well) and incubated over night at 4° C. Following the incubation, the plate was washed consecutively, by immersion, with de-ionized water and PBS (pH 6.5). The ELISA plate was then blocked with BSA (ICN Cat. No. 160069, 3%, 200 μl per well) for 1 hour at room temperature. Following blocking, the plate was washed with de-ionized water, then PBS (pH 6.5) plus Tween 20. CMV envelope glycoprotein B (Research Diagnostics, INC. Cat. No. RDI-RCMVAG-B) dissolved in PBS (supplemented with BSA (0.1%)) was incubated, separately, with heparin (Sigma Grade 1-A:From Porcine Intestinal Mucosa. Cat. No. H-3393). CMV envelope glycoprotein B (100 ng/ml) was incubated with a range of heparin concentrations (0.2-10 mg/ml) in a final volume of 100 μl, each concentration in duplicate for two hours at room temperature. Following the incubation, the samples were added to the BSA-blocked ELISA plate wells and incubated for two hours with shaking. Following incubation, the plate was washed with de-ionized water and PBS (pH 6.5) plus Tween. Mouse anti-human cytomegalovirus gB antibody (Research Diagnostics, INC. Cat. No. RDI-CMVG Babm) diluted in PBS (supplemented with BSA (0.1%)), 1:2000, was added to the ELISA plate (100 μl per well) and incubated for 90 minutes at room temperature with shaking. Following the incubation, the plate was washed with de-ionized water and PBS (pH 6.5) plus Tween. Goat anti-mouse IgG (H&L) Peroxidase Conjugated antibody (Chemicon International, Inc. Cat. No. AP124P) diluted in PBS (supplemented with BSA (0.1%)), 1:1000, was added to the ELISA plate (100 μl per well) and incubated for 30 minutes at room temperature with shaking. The peroxidase substrate chromogen, tetramethyl benzidine (TMB; Dako Cat. No. S1599) was added (100 μl per well) to the ELISA plate and incubated at room temperature. After 15 minutes, ELISA Stop Solution (hydrochloric acid 1N, sulfuric acid 3N) was added (200 μl per well) to stop the peroxidase catalyzed calorimetric reaction. The Optical Density (OD) of the samples was measured at 450 nm using an ELISA plate reader (Dynatech MR5000).

A dose response curve of soluble heparin inhibition of CMV envelope glycoprotein B binding to immobilized heparin is shown in FIG. 2.

### Example 3

An Assay for CMV Envelope Glycoprotein B Binding to GAGs that is suitable for the Screening of Compound Collections.

The GAGs, bovine kidney heparan sulfate (HS-GAG), shark cartilage chondroitin sulfate, hog skin dermatan sulfate, bovine cornea keratan sulfate and low molecular weight heparins, are commercially available (Sigma; Seikagaku Ltd, Japan). Human liver HS-GAG is purified as described (Dudas, J. et al., Biochem. J. 2000, 350, 245-251; Murata K., et al. 1985, Gastroenterology 89, 1248-1257). HS-GAG is conjugated to BSA to prepare a synthetic HS-GAG-BSA complex in which the HS-GAG is coupled via its reducing aldehyde terminus to the protein using sodium cyanoborohydride (Najjam, S. et al. 1997, Cytokine 12, 1013-1022). Other GAGs are coupled to BSA in a similar fashion.

The CMV envelope glycoprotein B (GBVP) binding assay is similar to the one described in Example 1. In brief, HS-GAG-BSA is added to a 96-well polystyrene ELISA plate and incubated over night at 4° C. Following the incubation, the plate is consecutively washed and blocked with BSA. GBVP, dissolved in PBS (supplemented with 0.1% BSA) is added to the ELISA plate and incubated for 60 minutes at room temperature with shaking. Following incubation, the plate is washed, incubated with antibody, washed and finally TMB is added to the ELISA plate. After 15 minutes, ELISA stop solution is added and the optical density of the samples is measured at 450 nm using an ELISA plate reader.

### Example 4

A Compound Screening Method—Contacting Test Compounds in the Presence of Heparin (or HS-GAG) and CMV Envelope Glycoprotein B, to Identify Inhibitor Compounds.

The CMV envelope glycoprotein B (GBVP) binding assay described in Example 1 was used to screen about 1,000 synthetic chemical compounds on 96-well plates. The compounds were purchased from ChemDiv Inc. (San Diego, Calif.). Compounds were dissolved in DMSO at 10 mM final concentration and further diluted prior to assay. DMSO concentration in the screening well was up to 2%. Individual compounds at a final concentration of 30 μM were co-incubated with GBVP on plates containing immobilized heparin and following washing, bound GBVP was detected with anti-CMV GBVP antibody and secondary antibody conjugated to horseradish peroxidase, as described in Example 1. Following color development, the % inhibition compared to control (no compound) for every compound was determined. Compounds that inhibited at least 30% of the signal were scored as hits. Examples of inhibitor compounds are listed in Table 1.

TABLE 1
Inhibition of CMV envelope glycoprotein B binding
to heparin by selected inhibitor compounds.

| Compound Number | % inhibition at 30 micromolar conc. |
|---|---|
| 1 | 34 |
| 2 | 52 |
| 3 | 93 |
| 4 | 58 |
| 5 | 64 |
| 6 | 47 |
| 10 | 90 |
| 11 | 70 |
| 25 | 80 |
| 26 | 50 |

### Example 5

An Assay to Demonstrate Direct Interaction of Inhibitor Compounds with Heparin and Other HS-GAGs.

In order to demonstrate that inhibitor compounds indeed bind directly to heparin and other HS-GAGs, individual compounds are incubated with immobilized heparin in the absence of GBVP. 96-well ELISA plates are coated with Heparin-BSA, then blocked with BSA as described in Example 1. GBVP Hit Compounds, at final concentration 0.1-200 μM, are incubated in the ELISA plate for 90 min, and then washed with incubation buffer. After washing, GBVP is added to the wells pre-incubated with compounds. At the same time, in separate control wells, GBVP is co-incubated with Hit Compounds for 90 min. Following the incubation, GBVP bound to the plate is quantified by antibody conjugated to horse radish peroxidase and OD measurement as described in Example 1. Examples of inhibitor compounds that interact directly with heparin are listed in Table 2.

TABLE 2
Direct binding of inhibitor compounds to heparin

| | Inhibition (%) | Inhibition (%) |
|---|---|---|
| Compound Number | Pre-Incubation | Co-incubation |
| 10 | 73 | 80 |
| 5 | 68 | 66 |
| 25 | 89 | 67 |

### Example 6

Inhibition of Varicella Zoster Virus Infectivity by Inhibitor Compounds

Cytopathic effect (CPE) inhibition assays were performed on Varicella Zoster Virus (VZV) as follows. Low passage human foreskin fibroblast (HFF) cells were seeded into 96-well tissue culture plates 24 hours prior to use at a cell concentration of 2.5×10⁵ cells per ml in 0.1 ml of MEM supplemented with 10% FBS. The cells were then incubated for 24 hours at 37° C. in a C0₂ incubator. After incubation, the medium was removed and 125 μl of candidate drug in growth medium (MEM with 10% FBS) was added to the first row in triplicate wells, all other wells containing 100 μl of media. The drug in the first row of wells was then diluted serially 1:5 throughout the remaining wells by transferring 25 μl using the Cetus liquid handling machine. After dilution of drug, 100 μl growth medium containing virus (2500 PFU per well) was added to each well, excluding cell control wells, which received 100 μl of growth medium only. The plates were then incubated at 37° C. in a CO₂ incubator for ten days. After the incubation period, media was aspirated and the cells stained with a 0.1% crystal violet solution for four hours. The stain was then removed and the plates rinsed using tap water until all excess stain was removed. The plates were allowed to dry for 24 hours and then read on a BioTek plate reader at 620 nm. Virus plaque numbers were used to determine the drug concentration required to inhibit viral replication by 50%, the effective concentration 50 (EC₅₀). Acyclovir (ACV) was used as a positive control drug.

A neutral red uptake assay was employed to determine the drugs' cytotoxicity. Twenty-four hours prior to the assay, HFF cells were plated into 96-well plates at a concentration of 2.5×10⁴ cells per well. After 24 h, the media,was aspirated and 125 μl of drug in growth medium was added to the first row of wells and then diluted serially 1:5 with growth medium using the Cetus liquid handling as in the CPE assay. After drug addition, the plates were incubated for seven days in a CO₂ incubator at 37° C. At this time, the media/drug was aspirated and 200 μl/well of 0.01% neutral red in PBS was added and the plates incubated in the CO₂ incubator for one hour. The dye was aspirated and the cells washed using a Nunc plate washer. After removing the PBS, 200 μl/well of 50% ETOH/1% glacial acetic acid (in H₂O) was added. The plates were rotated for 15 minutes and the optical densities (OD) read at 540 nm on a plate reader. The OD₅₄₀ₙₘ readings were used to determine the drug concentration required to inhibit 50% of stationary cells to take up neutral read, the cytotoxic concentration 50 (CC₅₀). (CPE and CC assays reference: Kern, E R. “Laboratory Procedures for Determining Antiviral Efficacy and Toxicity Against Herpes viruses and Orthopoxviruses”. Antiviral Research Laboratory. The University of Alabama at Birmingham, Department of Pediatrics, Division of Clinical Virology, BBRB 309; 1530 3^{rd} Avenue South, Birmingham, AL 35294-2170.)

It was found that inhibitor compound No. 10 had an EC₅₀=3.2 μg/ml in the CPE inhibition assay (ACV EC₅₀=0.37 μg/ml) and a CC₅₀=11.8 μg/ml in the cytotoxicity assay. It was found that inhibitor compound No. 11 had an EC₅₀<0.03 μg/ml (ACV EC₅₀=0.04 μg/ml) and a CC₅₀>100 μg/ml.

### Example 7

Inhibition of Bovine Viral Diarrhea Virus

Inhibitor compound 26 was tested on bovine viral diarrhea virus in MDBK (Madin-Darby bovine kidney cells; Yanagida K. et al., Antiviral Res. 2004 December; 64(3):195-201) cells and displayed excellent activity, EC₅₀=6 μM (IC₅₀>100 μM, SI>17). Bovine viral diarrhea virus is related to HCV and may serve as a surrogate model for human hepatitis C virus (Yanagida K., et al., ibid).

### Appendix

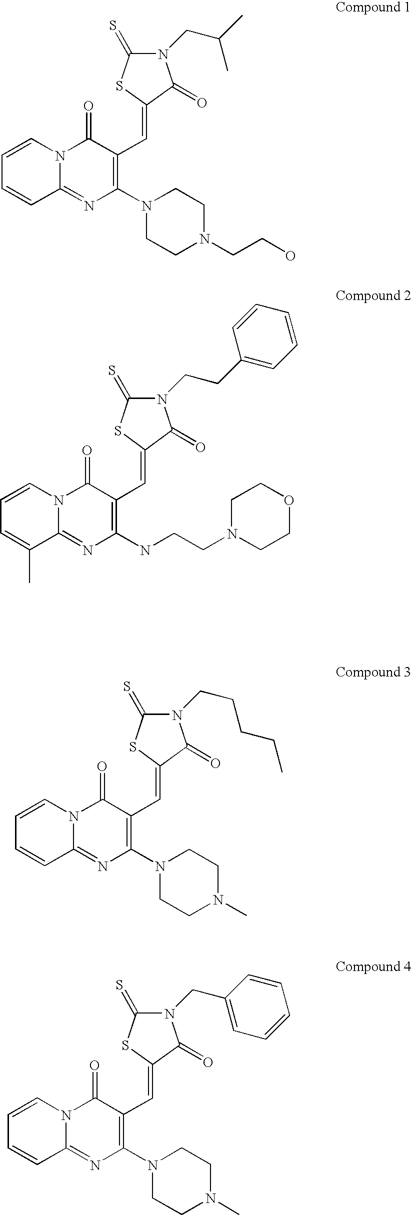

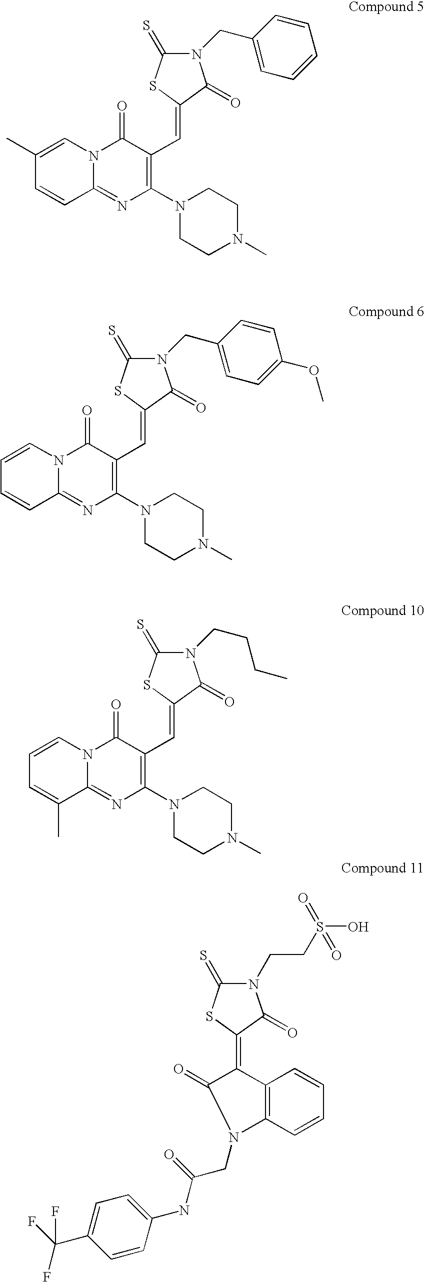

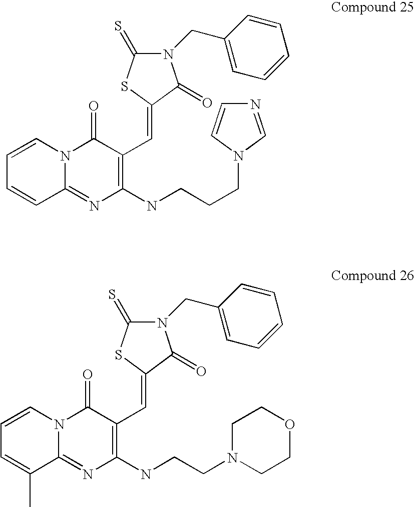

1. A method of screening for small organic molecules that directly inhibit the interaction of glycosaminoglycans (GAGs) with GAG-binding viral proteins (GBVPs), the method comprising the steps of:
(a) either contacting a GAG with an GBVP in the presence of at least one candidate compound; or contacting a GAG with at least one candidate small organic compound, removing unbound organic compound and adding a GBVP; and
(b) measuring the amount of the GAG bound to the GBVP or the amount of the GBVP bound to the GAG, wherein a significant decrease in GAG-GBVP binding as compared to GAG-GBVP binding in the absence of the candidate compound, identifies said compound as inhibitor of the GAG-GBVP interaction.

2. (canceled)

3. The method according to claim 1, wherein the GBVP is a fusion protein.

4. The method according to claim claim 1, wherein the GAG or the GBVP is tagged or labeled.

5. The method according to claim claim 1, wherein the GAG is heparan sulfate (HS-GAG) or heparin.

6. The method according to claim claim 1, wherein the small organic molecules are contacted with a proteoglycan containing GAG.

7. A method for the treatment or prevention of disorders related to virus attachment and entry or to bacterial or parasite attachment, comprising the step of administering to a subject in need thereof a therapeutically effective amount of a compound that directly inhibits the interaction of glycosaminoglycans (GAGs) with GAG-binding viral proteins (GBVPs), thus preventing virus attachment and entry or bacterial or parasite attachment mediated by the GAG.

8. The method according to claim 7, wherein the disorder related to virus attachment and entry is an infection caused by a virus selected from the group consisting of a HIV, a HSV, CMV, HCV, RSV, an influenza virus, and rhinovirus.

9. The method according to claim 8, wherein the disorder related to bacterial or parasite attachment is a bacterial infection or a parasite-induced disease such as malaria.

10. A pharmaceutical composition comprising a pharmaceutically acceptable diluent or carrier and an active ingredient of the general formula I: 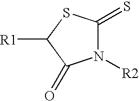 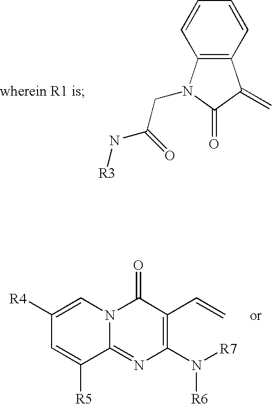
R2 is C₁-C₆ alkyl unsubstituted or substituted by a radical selected from the group consisting of —SO₃H, C₁-C₆ alkoxy, phenyl, 4-(C₁-C₆)alkylphenyl, 4-(C₁-C₆)alkoxyphenyl, 2-furyl, tetrahydro-2-furyl, or 1,3-benzodioxinyl, or R2 is cycloalkyl or C₂-C₆ alkenyl;
R3 is phenyl substituted by at least one radical selected from the group consisting of C₁-C₆ alkyl, hydroxy(C₁-C₆)alkyl, C₁-C₆ alkoxy, cyano, halogen, trifluoromethyl, cycloalkyl, aralkyl, aryl, substituted aryl, and heterocyclyl;
R4 and R5 each is hydrogen or C₁-C₆ alkyl; R6 and R7 each is selected from the group consisting of C₁-C₆ alkyl, C₁-C₆ alkyl substituted by piperidinyl, 4-morpholinyl, piperazinyl, 4-(C₁-C₆)alkyl-piperazinyl, 4-arylpiperazinyl, 4-aralkylpiperazinyl, or imidazolyl; C₃-C₇ cycloalkyl, C6-C₁₀ aryl, and C₇-C₁₆ aralkyl, or R₃ and R₄ together with the nitrogen atom to which they are attached form a 5 to 7 membered saturated heterocyclic ring containing one or two heteroatoms, or such 5 to 7 membered saturated heterocyclic ring containing an additional nitrogen atom substituted by C₁-C₆ alkyl or C₁-C₆ alkyl substituted by a radical selected from the group consisting of halogen, hydroxyl, C₁-C₆ alkoxy and phenyl, or by C₂-C₇ alkoxycarbonyl, and pharmaceutically acceptable salts thereof.

11. The pharmaceutical composition according to claim 10 comprising a compound of the general formula Ia: 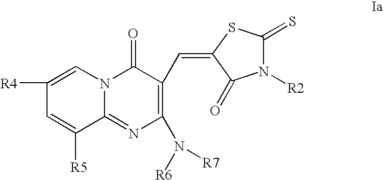
wherein:
R2 is C₁-C₆ alkyl unsubstituted or substituted by a radical selected from the group consisting of C₁-C₆ alkoxy, phenyl, 4-(C₁-C₆)alkylphenyl, 4-(C₁-C₆)alkoxyphenyl, 2-furyl, tetrahydro-2-furyl and 1,3-benzodioxinyl, or R2 is cycloalkyl or alkenyl;
R4 and R5 each is hydrogen or C₁-C₆ alkyl;
R6 and R7 each is selected from the group consisting of C₁-C₆ alkyl, C₁-C₆ alkyl substituted by piperidinyl, 4-morpholinyl, piperazinyl, 4-(C₁-C₆)alkyl-piperazinyl, 4-arylpiperazinyl, 4-aralkylpiperazinyl, or imidazolyl; C₃-C₇ cycloalkyl, C₆-C₁₀ aryl, and C7-C₁₆ aralkyl, or R₃ and R₄ together with the nitrogen atom to which they are attached form a 5 to 7 membered saturated heterocyclic ring containing one or two heteroatoms, or such 5 to 7 membered saturated heterocyclic ring containing an additional nitrogen atom substituted by C₁-C₆ alkyl or C₁-C₆ alkyl substituted by a radical selected from the group consisting of halogen, hydroxyl, C₁-C₆ alkoxy and phenyl, or by or C₂-C₇ alkoxycarbonyl,
and pharmaceutically acceptable salts thereof.

12. The pharmaceutical composition according to claim 11, wherein the compound of formula Ia is selected from the group consisting of:
4H-Pyrido[1,2-a]pyrimidin-4-one, 3-[[3-[(2-methylpropyl)methyl)]-4-oxo-2-thioxo-5-thiazolidinylidene]methyl]-2-[4-(2-hydroxyethyl)-1-piperazinyl]-(Compound 1)
4H-Pyrido[1,2-a]pyrimidin-4-one, 3-[[3-(phenylethyl)-4-oxo-2-thioxo-5-thiazolidinylidene]methyl]-2-[[2-(4-morpholinyl)ethyl]amino]-9-methyl-(Compound 2)
4H-Pyrido[1,2-a]pyrimidin-4-one, 3-[(3-pentyl -4-oxo-2-thioxo-5-thiazolidinylidene)methyl]-2-(4-methyl-1-piperazinyl)-(Compound 3)
4H-Pyrido[1,2-a]pyrimidin-4-one, 3-[[3-(phenymlethyl)-)-4-oxo-2-thioxo-5-thiazolidinylidene]methyl]-2-(4-methyl-1-piperazinyl)-(Compound 4)
4H-Pyrido[1,2-a]pyrimidin-4-one, 3-[(3-phenylmethyl-4-oxo-2-thioxo-5-thiazolidinylidene)methyl]-2-(4-methyl-1-piperazinyl)-7-methyl-(Compound 5)
4H-Pyrido[1,2-a]pyrimidin-4-one, 3-[[3-[(4-methoxyphenyl)methyl]-4-oxo-2-thioxo-5-thiazolidinylidene]methyl]-2-(4-methyl-1-piperazinyl)-(Compound 6)
4H-Pyrido[1,2-a]pyrimidin-4-one, 3-[(3-butyl-4-oxo-2-thioxo-5-thiazo-lidinylidene)methyl]-9-methyl-2-(4-methyl-1-piperazinyl)-(Compound 10)
4H-Pyrido[1,2-a]pyrimidin-4-one, 3-[(3-phenylmethyl-4-oxo-2-thioxo-5-thiazolidinylidene)methyl]-2-[[3-(1H-imidazol-1-yl)propyl]amino]-(Compound 25)
4H-Pyrido[1,2-a]pyrimidin-4-one, 3-[[3-(phenylmethyl)-4-oxo-2-thioxo-5-thiazolidinylidene]methyl]-2-[[2-(4-morpholinyl)ethyl]amino]-9-methyl-(Compound 26).

13. The pharmaceutical composition according to claim 10, comprising a compound of the general formula Ib: 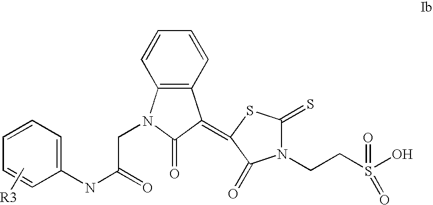
wherein:
R3 is C₁-C₁₀ alkyl, hydroxy(C₁-C₁₀)alkyl, C₁-C₆ alkoxy, cyano, halogen, trifluoromethyl, cycloalkyl, aralkyl, aryl, substituted aryl, and heterocyclyl;
and pharmaceutically acceptable salts thereof.

14. The pharmaceutical composition according to claim 13, wherein R3 is methyl, ethyl, hydroxyethyl, halogen, cyano, 3,4-dicyano, methoxy, 4,5-dimethoxy, or 3-trifluoromethyl.

15. The pharmaceutical composition according to claim 13, wherein the compound of formula Ib is:
5-[1,2-dihydro-2-oxo-1-[2-oxo-2-[[3-(trifluoromethyl)phenyl]amino]ethyl]-3H-indol-3-ylidene]-4-oxo-2-thioxo-3-thiazolidineethanesulfonic acid [Compound 11]; or
5-[1,2-dihydro-2-oxo-1-[2-oxo-2-[3-(cyanophenyl)amino]ethyl]-3H-indol-3-ylidene]-4-oxo-2-thioxo-3-thiazolidine ethanesulfonic acid.

16. The pharmaceutical composition according to claim 10, for treatment or prevention of viral diseases, disorders or conditions mediated by virus-to-cell attachment via heparan sulfate glycosaminoglycans (HS-GAGs).

17. The pharmaceutical composition according to claim 16, wherein the viral disease is an infection caused by a virus selected from the group consisting of a HIV, a HSV, CMV, HCV, RSV, an influenza virus, and rhinovirus.

18. The pharmaceutical composition according to claim 10, for treatment or prevention of disorders mediated by bacteria-to-cell or parasite-to-cell attachment via HS-GAGs.

19-21. (canceled)

22. A method for the treatment or prevention of viral diseases, disorders or conditions mediated by virus-to-cell attachment via HS-GAGs, comprising the step of administering to a subject in need thereof a therapeutically effective amount of a pharmaceutical composition comprising a compound of the general formula I in claim 10.

23. The method according claim 22, wherein the viral disease is selected from a group consisting of HIV, HSV, CMV, HCV, RSV, influenza virus, and rhinovirus infection.
